(19) United States
(12) Patent Application Publication
(10) Pub. No.: US 2008/0038194 A1
(43) Pub. Date: Feb. 14, 2008

(54) System and Method for Expression Proteomics Based on Isotope Ratio Modification

(75) Inventors: Julian P. Whitelegge, Costa Mesa, CA (US); David B. Agus, Beverly Hills, CA (US); Jonathan Katz, Encino, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 10/597,054

(22) PCT Filed: Jan. 13, 2005
(86) PCT No.: PCT/US05/01393

§ 371 Date: Jul. 2, 2007

Related U.S. Application Data
(60) Provisional application No. 60/536,766, filed on Jan. 15, 2004.

(51) Int. Cl. A61B 5/055; A61K 51/00; A61P 43/00; C07C 331/16

(52) U.S. Cl. 424/1.69; 424/9.1; 436/86

(57) ABSTRACT

The present invention relates to modification of isotope ratios in peptides and polypeptides as an alternative to full isotopic exchange for coding in proteomics. Subtle modification of isotope ratio does not compromise protein identification experiments and can thus provide elemental composition data for accurate isotope ratio decoding. Application of subtle modification of isotope ratio proteomics (SMIRP) offers a convenient approach to in vivo isotope coding.

### FIELD OF THE INVENTION

The invention relates to expression proteomics, and particularly, to a system and method for analyzing expression proteomics based on a modification or modifications to isotope ratio.

### BACKGROUND OF THE INVENTION

Proteomics seeks to monitor the flux of protein through a biological system under variable developmental and environmental influences as programmed by the genome (Whitelegge, “Plant proteomics: BLASTing out of aMudPIT,” Proceedings of the National Academy of Sciences of the United States of America, Session 99, pp. 11564-1566 (2002)). Consequently, it is necessary to measure changes in protein abundance and turnover rate as faithfully as possible. In the absence of non-invasive technologies, the majority of proteomics approaches involve destructive sampling at various time points to obtain ‘snapshots’ that periodically report the genome's product. Thus, quantitation has become the major challenge facing the field as it matures. Because of the variability of day-to-day measurements of protein quantities, a common feature of quantitative proteomics is the use of stable isotope coding to distinguish control and experimental samples in a mixture that can be profiled in a single experiment (Gygi et al., “Quantitative analysis of complex protein mixtures using isotope-coded affinity tags,” Nature Biotechnology, Vol. 17, pp. 994-999 (1999); Conrads et al., “Current strategies for quantitative proteomics,” Advances in Protein Chemistry, Vol. 65, pp. 133-159 (2003)). Isotope coding was introduced to allow the distinction between two samples in a single mass spectrometry experiment in order to achieve improved relative quantitation since run-to-run variability is eliminated (Tao and Aebersold, “Advances in quantitative proteomics via stable isotope tagging and mass spectrometry.,” Current Opinions in Biotechnology, Vol. 14, pp. 110-18 (2003)). Both chemical modification (Gygi et al., 1999) and in vivo labeling approaches (Pasa-Tolic et al., “High throughput proteome-wide precision measurements of protein expression using mass spectrometry,” Journal of the American Chemical Society, Vol. 121, pp. 7949-7950 (1999)) have been successful. In vivo pulse-chase experiments with stable isotopes allow for measurement of protein turnover rates (Pratt et al., “Dynamics of protein turnover, a missing dimension in proteomics,” Molecular and Cellular Proteomics, Vol. 1, pp. 579-591 (2002); Whitelegge et al., “Mass Spectrometry of the Lactose Permease,” Transmembrane Transporters, Receptor Biochemsitry and Methodology Series, M. Quick, ed. pp. 179-90 (2002)). Coding with stable isotopes can be achieved by growth of an organism in depleted/enriched media or by chemically modifying proteins after extraction from the organism. There are a number of stable isotope strategies currently used to study biological systems. Each of these strategies is predicated on the use of a complete or near-complete isotope swap in the molecules, sample or entire organism being analyzed. This process is sometimes referred to as “conversion” with reference to the molecules, sample, or entire organism.

In general, a non-natural isotope is “swapped” for the naturally occurring variety. For instance, ¹⁵N may be swapped for ¹⁴N, ¹³C for ¹²C, or ¹⁸O for ¹⁶O, such that a peptide's mass is altered by several Daltons. A complete (or near-complete) isotope swap may be achieved by any number of strategies well known to those of skill in the art. By way of example, these strategies may include isotope-coded affinity tag (“ICAT”) technology, stable isotope labeling with amino acids in cell culture (“SILAC”), enzymatic molecular introduction (i.e., exchange) of ¹⁸O, and growth of a biological sample, a cell culture, or an entire organism in stable isotopes. By inducing an isotope swap of this nature, it becomes possible to examine a range of biological processes at the molecular level.

After the development of isotope ratio mass spectrometers, it was shown that ¹³C was naturally depleted in plants and that the extent of depletion varied among plants (Nier and Gulbransen, “Variations in the relative abundance of the carbon isotopes,” Journal of the American Chemical Society, Vol. 61, pp. 697-698 (1939); Murphy and Nier, “Variations in the relative abundance of the carbon isotopes,” Physical Review, Vol. 59, pp. 771-772 (1941)). ¹³C isotope depletion measurements were integral to the discovery of C4 photosynthesis and crassulacean acid metabolism CAM; C3 plants are ¹³C depleted by about 30% compared to 10-15% in C4 and 10-25% for CAM plants. There are several origins of ¹³C depletion including thermodynamic fractionation by the carboxylation activity of ribulose-bisphosphate carboxylase/oxygenase (RUBISCO, 29% depletion) and various physical contributions related to solubility, diffusion and hydration of CO₂ in water combined with biological bias from enzymes that impinge upon these properties. Furthermore, while isotope fractionation during small molecule flux is quite well understood, less is known about the process with respect to protein synthesis. Recent literature has reported minor variations in isotope ratio for different amino acids from specific protein sources as well as positional effects within amino acids (Jim et al., “Effects of hydrolysis on the delta ¹³C values of individual amino acids derived from polypeptides and proteins,” Rapid Communications in Mass Spectrometry, Vol. 17, pp. 2283-2289 (2003); Sacks and Brenna, “High-precision position-specific isotope analysis of 13C/12C in leucine and methionine analogues,” Analytical Chemistry, Vol. 75, pp. 5495-5503 (2003)). ¹⁸O bias has been reported in plants and has the potential to interfere with calculation of accurate ¹³C/¹²C ratios when the second “¹³C” isotope peak is included in calculation of isotope ratio (Schmidt et al., “¹⁸O pattern and biosynthesis of natural plant products,” Phytochemistry, Vol. 58, pp. 9-32 (2001)). Minor contributions from D/H and ¹⁵N /¹⁴N fractionation might also contribute minor variability.

While isotope swapping technology has important implications and uses in the study of molecular biology, it is significantly limited in its applications. A number of disadvantages have emerged, including isotope effects upon retention during liquid chromatography (Zhang et al., “Fractionation of isotopically labeled peptides in quantitative proteomics,” Analytical Chemistry, Vol. 73, pp. 5142-5149 (2001)), loss of separation space in the mass spectrometer and expense. Furthermore, it is generally not applicable, for example, to the study of living humans and other animals. Although isotopically modified animals have been produced (Wu et al., “Metabolic Labeling of Mammalian Organisms with Stable Isotopes for Quantitative Proteomic Analysis,” Analytical Chemistry, Vol. 76, Issue 17, pp. 4951-9 (2004)), inducing a complete swap of isotopes in living humans—even if it could be achieved, which is by no means a simple assumption—is not likely to be a viable option for a number of reasons including significant health concerns and the tremendous expense and time required to effect such a change. Unlike the labeling of bacteria and plants, in which the entirety of the carbon content can be derived from CO₂ with concomitant isotopic labeling of all the polypeptides, the labeling of the polypeptides of animals is not as simple. The most straightforward way to manipulate the carbon isotopic ratio in animals would be by incorporating ¹³C-enriched amino acids into the diet. Unfortunately, because of the differential flux of free amino acid pools, it is quite possible that polypeptides will not be homogeneously labeled. For example, the concentrations of the branched chain, aromatic and sulfur containing amino acids in humans are only minimally affected by dietary supplement (Millward and Rivers, “The nutritional role of indispensible amino acids and the metabolic basis for their requirements,” European Journal of Clinical Nutrition, Vol. 42, pp. 367-393 (1988)). A more direct comparison was done that shows that supplementation by both leucine and lysine at similar concentrations leads to a 2-fold increase in lysine concentration over that of leucine even though both amino acids are found in similar abundance in proteins and would therefore be expected to have the same metabolic flux (Bergstrom et al., “Effect of a test meal, without and with protein, on muscle and plasma free amino acids,” Clinical Science, Vol. 79, pp. 331-337 (1990)). Furthermore, performing polypeptide analysis based on partial ¹³C labeling via dietary amino acids is complicated by the many amino acids that are biochemically close to core metabolic pathways that would be expected to undergo rapid isotopic shuffling. For example, alanine and glutamate can undergo a transamination to become pyruvate and ketoglutarate, respectively; in a study of the incorporation of dietary labeled amino acids in chicken feed into egg protein only 11% of the alanine and 7% of the glutamate were incorporated without metabolic transformation (Berthold et al., “Uniformly ¹³C-labeled algal protein used to determine amino acid essentiality in vivo,” Proceedings of the National Academy of Sciences of the United States of America, Session 88, pp. 8091-8095 (1991)). Additionally, as might be expected, methionine partially behaved as an amino acid containing one less carbon due to methionine's role in methyl transfer biochemistry. These differences in flux suggest that studies in higher eukaryotes might be best performed by labeling the amino acids that show the lowest turnover.

These methods are expensive because of the need for high isotope purity. Furthermore, the fact that two peptide isotope distributions replace one leads to a practical loss of separation space in the mass spectrometer demanding more efficient peptide separations. Moreover, the second isotope distribution may trigger MSMS in proteomics experiments wasting mass spectrometer time. Thus, in order to implement isotope swapping techniques in the study of such systems, a different methodology is believed to be required. Such a methodology may have a dramatic impact on the study of biology and biological systems, and particularly human and animal biology, as well as other systems that may not be appropriate for study in connection with the conventional methods described above.

### SUMMARY OF THE INVENTION

Described herein are a system and method for subtly modifying isotope distribution for expression proteomics. The system and method include administering to an organism a composition with a modified isotope profile. The invention further includes monitoring the turnover of identified peptide sequences.

In another embodiment of the present invention, a process for decoding isotope distribution is provided. In various embodiments of the present invention, the isotope ratio of particular peptides or polypeptides in an organism is modified. Various molecules may be modified ¹³C for ¹²C, ¹⁸O for ¹⁶O, ¹⁵N for ¹⁴N and the like. The isotope ratio of ¹³C:¹²C may, for example, be modified from a ratio of 100:1 to 200:1. The isotope modification of the present invention may be implemented by various means including, but not limited to, ICAT, SILAC, enzymatic exchange, growth in stable isotopes, diet and injection.

### BRIEF DESCRIPTION OF FIGURES

FIG. 1 depicts spectrographs of altered isotope profiles of peptides from cells grown in media with altered ¹³C/¹²C ratio in accordance with an embodiment of the present invention. The spectra are MALDI-TOF spectra of a tryptic peptide from phycocyanin A obtained from cultures of Synechocystis sp. PCC 6803 grown under elevated ¹³C. The spectra show increased abundance of isotopic species containing ¹³C.

FIG. 2 depicts spectrographs of altered isotope profiles of peptides from cells grown in media with altered ¹³C/¹²C ratio in accordance with an embodiment of the present invention. The ion-trap ESI spectra were recorded on an ion-trap mass spectrometer showing spectra of the same peptide as in FIG. 1. The spectra are single scans from a ‘triple-play’ protein identification experiment. The relative abundance of the isotopomers yielding ¹³C/¹²C ratio potentially provides relative expression measurement in parallel with protein identification.

FIG. 3 demonstrates performance of protein identification by tandem mass spectrometry under conditions of modified ¹³C/¹²C isotope ratio in accordance with an embodiment of the present invention. Tandem mass spectra from the ‘triple-play’ experiment shown in FIG. 2 are shown for control (a) and 3% samples (b), both derived from the doubly charged parent. FIG. 3 shows a notable shift of higher mass b and y fragments in (b). Increased abundance of ¹³C isotope containing species did not affect protein identification. Performance was compromised in the 6% sample (not shown). Successful peptide identification allows the use of elemental composition in calculation of ¹³C/¹²C ratio.

### DESCRIPTION OF THE INVENTION

The present invention is based on a novel approach to the study of molecular biology, by inducing subtle modifications in the isotope ratio of particular peptides and polypeptides for expression proteomics. “Subtle,” as used herein with reference to the modification of isotopes included in target molecules, is defined as a “swapping” of, on average, an amount of isotopes included in target molecules such that there is a measurable effect upon the observed peptide isotope distribution, without causing a gross extension or displacement of the single isotope envelope. The modification introduced is gross compared to natural isotopic variability yet subtle compared with strategies that seek full exchange. Isotope ratio is calculated for specific peptides or polypeptides based upon their isotopic distributions obtained by high-resolution mass spectrometry. This requires either an estimate of elemental composition based upon mass and average amino acid elemental composition (averagine), or the precise elemental composition based upon the peptide sequence determined by tandem mass spectrometry and protein identification algorithms, as in typical proteomics experiments. Successful implementation of the present invention was demonstrated by modest elevation of the ¹³C/¹²C ratio in Synechocystis sp. PCC 6803 cultures as a model for other organisms. Small fluctuations of isotope ratios occur in living organisms as a result of metabolic bias and nutrition (Meier-Augenstein et al., “Applied gas chromatography coupled to isotope ratio mass spectrometry,” Journal of Chromatography, Vol. 842, pp. 351-371 (1999)). In this model, carbon was supplied as CO₂/HCO₃ (from bicarbonate) and entered the metabolic cycle via carbon fixation.

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), provides one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. Alternate methodologies and procedures may be readily implemented without undue experimentation. The presently disclosed embodiments are therefore to be considered in all respects as illustrative and not restrictive. Example methodologies and procedures may be readily understood by reference to the Results and Examples described below. Furthermore, all publications referred to herein are incorporated in their entirety.

Subtle modifications may be induced with reference to any suitable target isotope or isotopes, which may include, but are in no way limited to, ¹³C for ¹²C, ¹⁸O for ¹⁶O, and ¹⁵N for ¹⁴N. Additional isotopes suitable for subtle modification in accordance with alternate embodiments for the present invention will be readily appreciated by those of skill in the art; for instance, deuterium may be swapped for hydrogen.

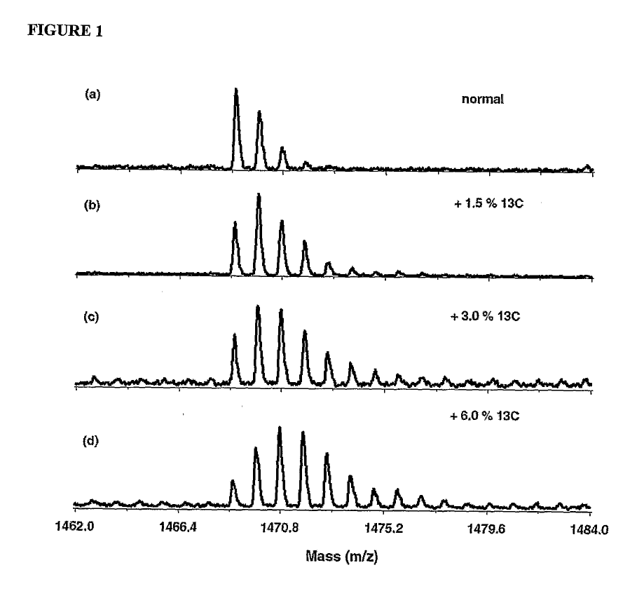

Subtle alteration of the ratio of ¹³C to ¹²C may be particularly advantageous in connection with the methods of the present invention, insofar as these methods are implemented in connection with proteomics. Carbon is the most abundant constituent of proteins, and, thus, a small change in the ratio of ¹³C to ¹²C has the most dramatic effect upon isotopic distribution; changing this ratio from 100:1 to 100:2 (or 200:1), for instance, may have quite a dramatic effect. In the context of proteomics, this subtle alteration of isotopic ratio can be measured from the isotopic distribution of peptide ions; thereby providing a means of stable isotope tagging that does not require full conversion to a non-natural isotope. For example, FIG. 1 illustrates a MALDI readout from Synechocystis cultures in which subtle modifications of target molecules were induced (normal IR, +1.5%, +3.0% and +6% ¹³C, respectively; from top to bottom of Figure), in accordance with an embodiment of the present invention.

The methods of the present invention are by no means limited to the study of proteomics, however. In fact, the invention may find application in a host of biological systems, as well as non-biological systems (i.e., in the study of any system in which isotopes may be subtly modified and thereafter analyzed by the methods described herein).

Methods for inducing the subtle modifications incorporated in various aspects of the present invention will similarly be recognized by and may be readily implemented by those of skill in the art without undue experimentation. In addition to the use of variants of the technology described above (i.e., ICAT, SILAC, enzymatic exchange, and growth in stable isotopes), other means for inducing the subtle modification of isotopes can be readily ascertained. For example, a living animal may be fed a diet that includes the non-natural isotope or isotopes sought to be introduced into their internal physiology; diet influences actual isotope ratios in animals, including humans. Such a diet may include, for example, food (e.g., animal chow) supplemented with the non-natural isotope or, in another embodiment of the invention, the diet may include deuterated or deuterium-enriched water. Alternatively, the isotope may be introduced by pharmacological means (e.g., a diet supplement), or by other conventional forms of administration (e.g., injection of saline consisting of the non-natural isotope). The particular mode of adminstration may be selected based upon the physiological process to be studied.

Once the target molecule (or molecules) has been subtly modified, it may be studied by a number of different technologies. For example, isotope ratio mass spectrometry (“IRMS”) may be employed, whereby levels of different isotopes (e.g., ¹³C and ¹²C) may be measured after conversion to carbon dioxide (e.g., by combustion). Alternatively, the isotope ratio may be measured by calculation from the peptide mass spectra obtained by various forms of mass spectrometry (e.g., MSMS). Specific protein molecules can be identified by MSMS in connection with an appropriate database search, or isotopic distribution can be estimated using averagine (i.e., a model amino acid with elemental components occurring at frequencies deduced from the PIR database). In particular embodiments of the invention, high resolution mass spectrometry, such as Fourier transform mass spectrometry (“FTMS”), may be particularly advantageous and accurate. Moreover, in instances where peptide sequences (and, thus, elemental compositions) are known, the measurements attainable on high-resolution instrumentation, such as mass spectrometers employing FTMS, may be highly accurate.

The present invention has a range of applications. In one embodiment, the invention may be used in connection with isotope coding by subtle alteration of isotope ratio in proteins, peptides and polypeptides. This may be particularly useful in connection with proteomics. For instance, one may study the relative expression of various proteins in a biological system. Two (or more) samples can be distinguished by their isotope ratios; thereby allowing mixing and relative expression measurement by comparison of peak height/areas (i.e., in a MALDI readout). The isotope ratio of a peptide in a mixture is determined by relative contribution from non-labeled as compared with labeled material.

In another embodiment, the present invention may be used to study protein turnover (e.g., one may monitor metabolic or transcriptional activity by seeking out newly altered or transcribed proteins, respectively). Protein turnover may be measured using pulse-chase protocols. For instance, if a human begins to eat food with an altered isotope ratio, then this will first be observed in rapidly turning-over proteins. Slow-turning proteins will be last to manifest the change in isotope ratio.

Among the benefits of employing the methods of the present invention in connection with, for example, proteomics, is the significant cost savings and the ability to study systems that were heretofore impossible to study by way of isotope swapping.

The ¹³C/¹²C isotope ratio of Synechocystis sp. PCC 6803 grown in culture was altered modestly via manipulation of the source of CO₂ for photoautotrophic growth (bicarbonate). Soluble proteins were separated from the membrane fraction after mechanical disruption of the cells. Membrane proteins were precipitated with acetone, dissolved in formic acid and subjected to analysis by liquid chromatography electrospray-ionization mass spectrometry with fraction collection (LC-MS+). Fractions were reduced, alkylated and digested with trypsin prior to MALDI-TOF analysis. FIG. 1 compares the isotopic distribution for a peptide derived from phycocyanin A from control and the ¹³C supplemented cultures. The change in isotopic distribution is readily apparent from the spectra and ¹³C/¹²C ratio was calculated from peak heights and areas using the Isosolv algorithm. The Isosolv algorithm, explained further in Example 7, measures carbon isotope distribution. Specifically, the probability of having ‘n’ ¹³C's given ‘X’ total carbons and a ¹³C probability of ‘P’ is based on the following formula:

prob(n)=combin(X,n)*Pn*(1−P)(X−n).

Given an elemental composition, Isosolv is used to estimate ¹³C/¹²C ratio. For any particular molecular weight, the number of carbons is estimated by dividing the molecular weight by 110 (the average mass of an amino acid) and multiplying by 4.94 (the average number of carbons per amino acid). In turn, given a measured isotopic distribution, the ¹³C probability can be determined by calculating the difference between the measured distribution and the theoretical distribution for an arbitrary ¹³C abundance. The estimated ¹³C abundance parameters can then be incrementally altered until the error between the theoretical distribution and the calculated distribution has been minimized, thereby yielding the ¹³C probability in the measured spectrum.

Table 1 summarizes the ¹³C/¹²C isotope ratios of peptides derived from their isotopic distribution^{a}.

| | | Ion-trap^{d} | Ion-trap^{d} | Ion-trap | Ion-trap |
|---|---|---|---|---|---|
| % ¹³C | | M + H⁺ | M + H⁺ | M + 2H⁺ | M + 2H⁺ |
| added^{b} | MALDI-TOF^{c} | (height^{e}) | (area^{e}) | (height^{e}) | (area^{e}) |
| 0 | 0.0093 | 0.0114 | 0.0115 | 0.0105 | 0.0117 |
| | 0.0107 | 0.0141 | 0.0116 | 0.0092 | 0.0076 |
| | 0.0099 | 0.0134 | 0.0112 | 0.0143 | 0.0124 |
| | 0.0104 | 0.0136 | 0.0114 | 0.0119 | 0.0105 |
| Mean | 0.0101 | 0.0131 | 0.0114 | 0.0115 | 0.0106 |
| SD | 0.0006 | 0.0012 | 0.0002 | 0.0022 | 0.0021 |
| 1.5 | 0.0199 | 0.0178 | 0.0167 | 0.0148 | 0.0125 |
| | 0.0196 | 0.0180 | 0.0150 | 0.0117 | 0.0125 |
| | 0.0200 | 0.0164 | 0.0156 | 0.0164 | 0.0136 |
| | 0.0199 | 0.0172 | 0.0156 | 0.0175 | 0.0167 |
| Mean | 0.0199 | 0.0174 | 0.0157 | 0.0151 | 0.0138 |
| SD | 0.0002 | 0.0007 | 0.0007 | 0.0025 | 0.0020 |
| 3 | 0.0272 | 0.0288 | 0.0250 | 0.0244 | 0.0230 |
| | 0.0261 | 0.0281 | 0.0266 | 0.0277 | 0.0224 |
| | 0.0274 | 0.0305 | 0.0253 | 0.0225 | 0.0218 |
| | 0.0293 | 0.0276 | 0.0247 | 0.0241 | 0.0222 |
| Mean | 0.0275 | 0.0288 | 0.0254 | 0.0247 | 0.0224 |
| SD | 0.0013 | 0.0013 | 0.0008 | 0.0022 | 0.0005 |
| 6 | 0.0399 | 0.0358 | 0.0346 | 0.0362 | 0.0328 |
| | 0.0400 | 0.0355 | 0.0319 | 0.0396 | 0.0330 |
| | 0.0324 | 0.0344 | 0.0383 | na^{f} | na |
| | 0.0398 | 0.0341 | 0.0315 | na | na |
| Mean | 0.0380 | 0.0350 | 0.0341 | 0.0379 | 0.0329 |
| SD | 0.0038 | 0.0008 | 0.0031 | 0.0024 | 0.0001 |

^{a}The Isosolv algorithm (experimental) was used to calculate ¹³C/¹²C ratio for the peptide FLSSTELQIAFGR (amino acids 18-30 phycocyanin A; Q54715; elemental composition C67 H106 N17 O20; calculated M + H⁺ 1468.7794 Da.
^{b}Targeted ¹³C addition.
^{c}Sum of 300-500 laser flashes.
^{d}LCQ-DECA single zoom scan data.
^{e}Use of peak height versus area for calculation of isotope ratio.
^{f}Not available.

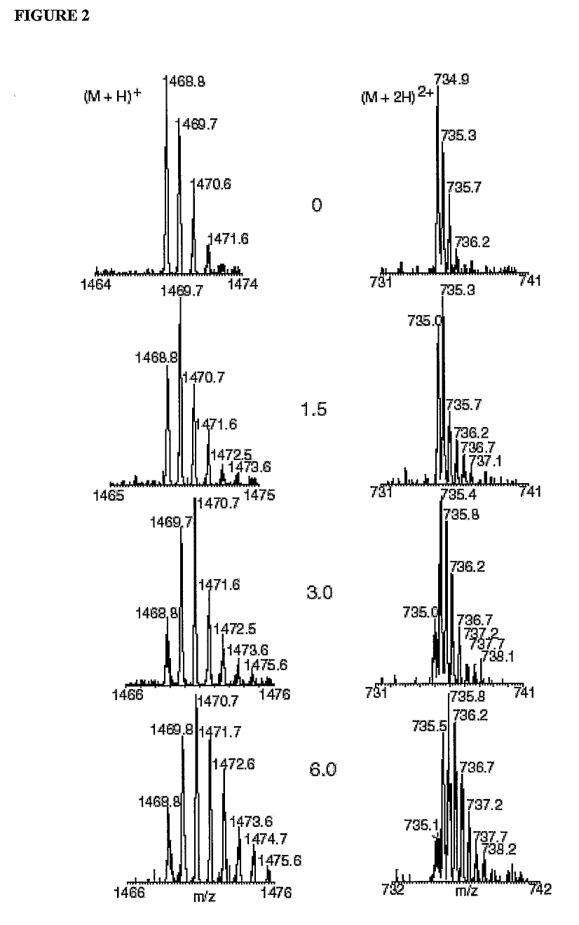

There was a general decrease in signal to noise in the higher supplementation samples (see FIGS. 1(c) and 1(d)) with signals appearing at every unit across the mass range. This makes identification of the monoisotopic peak problematic and points to the benefits of subtle modification of isotope ratio. FIG. 2 shows the same peptide from LC-MSMS analysis of reduced, alkylated and trypsinized proteins from the Synechocystis sp. samples. For this experiment, the zoom scan feature of the ion-trap mass spectrometer operating in data-dependent acquisition mode (obtained from ThermoElectron; LCQ-DECA) was used for typical protein identification experiments, where ions are excluded from MSMS analysis when a zoom scan (10 Da width) shows them to be singly charged. Both single and doubly charged ions are shown, again displaying the clearly altered isotopic distributions, as in FIG. 1. It should be noted that at higher ¹³C supplementation the isotopic envelope for the single peptide was widened considerably, contributing to loss of separation space in the mass spectrometer; again subtle modification of ¹³C/¹²C ratio appears desirable. The doubly charged ions showed broadly comparable isotopic distributions to the singly charged ions with greater experimental variability apparent. The ¹³C/¹²C ratios calculated for these spectra are shown in Table 1 and compared to the results from MALDI-TOF. In the case of MALDI and zoom scans on singly charged ions by ion trap, the ¹³C/¹²C ratios calculated are sufficient to distinguish the samples from each other, that is, they have been successfully isotope coded. However, the performance was less satisfactory for the doubly charged ions. Considerable variability was observed between zoom scans on the same peptide (see Table 1) presumably resulting from experimental variability of isotopomer capture in the ion-trap experiment. Use of peak area as opposed to height reduced measurement variability. Increasing the number of scans would improve statistics but is undesirable in the context of the proteomics experiment. Similarly, improvements in instrument performance with respect to accuracy, resolution and sensitivity will all improve reproducibility. Evaluation of next generation of linear ion-traps and Fourier-transform instruments may be helpful in this respect. The discrepancy between measured isotope ratio and the theoretically targeted ratio conferred by the bicarbonate is under investigation, but may be due to exchange of CO₂ with the atmosphere during the experiment. The cultures cannot be sealed due to the need to allow photosynthetically produced O2 to escape.

It was desirable that the isotope coding strategy be compatible with existing protein identification protocols, thus, to that end, the protein identification performance was examined in the LC-MSMS experiments used for FIG. 2 (MSMS on the doubly charged ions only).

Table 2 summarizes performance of the experiment when interrogated with Sequest for protein identifications. Protein identification performance was observed under altered ¹³C/¹²C ratios^{a}.

| % | Tryptic | Tryptic | | |
|---|---|---|---|---|
| ¹³C | peptides | peptides | Peptides | ΔCn^{c} |
| added | (Xcorr^{b} > 2.3) | (Xcorr > 4.0) | Phycocyanin B | Phycocyanin B |
| 0 | 43 | 11 | 17 | 164.26 |
| 1.5 | 40 | 15 | 15 | 142.26 |
| 3.0 | 40 | 17 | 16 | 160.26 |
| 6.0 | 25 | 10 | 11 | 110.27 |

^{a}Protein identification used Sequest (ThermoFinnigan) to match experimental tandem mass spectrometry data to a database of translated Synechocystis sp. PCC 6803 open reading frames. ‘No enzyme’ is selected so that all possible sequences are screened. The results of a representative experiment are shown.
^{b}The cross-correlation coefficient (Xcorr) provides a measure of how well a tandem mass spectrum matches that predicted for a particular peptide. Searches that yield tryptic peptide matches with Xcorr > 2.3 are generally significant matches. Searches that yield tryptic peptide matches with Xcorr > 4.0 are nearly always highly significant matches with good signal to noise.
^{c}The delta correlation (ΔCn) is a measure of how well a number of peptide matches identify a specific protein.

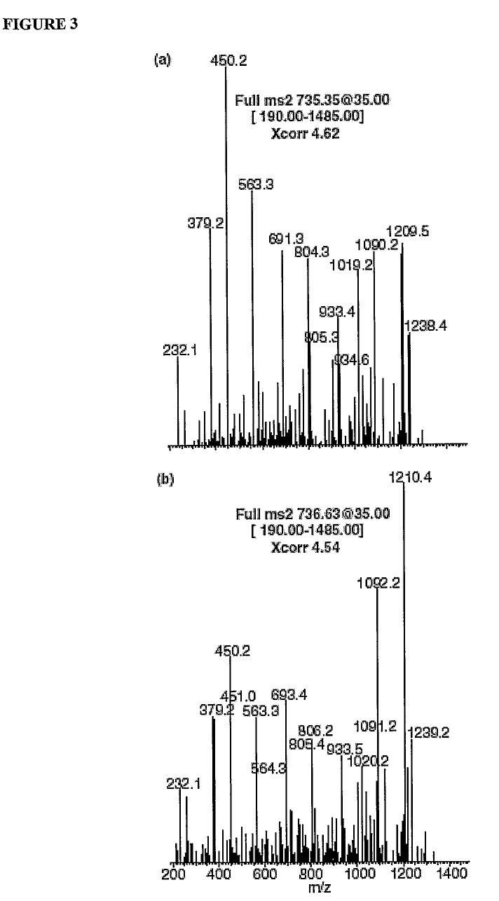

It is noted that the lower ¹³C supplemented samples were unaffected with respect to peptides/proteins identified while a noticeable decline was observed for the highest ¹³C sample. The widening of the isotopic envelope may lead to less frequent triggering of the MSMS experiment as maximum signal intensities drop. Also MSMS spectra may fail to yield Sequest hits as higher mass b and y fragments become too large to fall within tolerance limits for database matching. In FIG. 3, the MSMS spectra of the same 1468 Da peptide from Phycocyanin A (doubly charged parent) are compared for the control and the 3.0% supplementation samples. It is noted that while Sequest returned strong correct identifications for both peptides, the dominant larger b and y fragments in the 3.0% sample exhibit the mass of the first ¹³C isotope rather than the monoisotopic mass. It is concluded that Sequest remains efficient until the monoisotopic species is sufficiently diminished in abundance at the highest ¹³C supplementation. Furthermore, it is concluded that the standard performance of Sequest tolerates subtle modification of isotope ratio in ranges that are useful for isotopic coding (+0.75-1.5% supplementation; 750-500%).

Modification of ¹³C/¹²C was chosen because carbon is the most abundant element in peptides and proteins, though the strategy could also employ ¹⁵N:¹⁴N or ¹⁸O:¹⁶O manipulation as discussed above. Cargile (Cargile et al., “Synthesis/degradation ratio mass spectrometry for measuring relative dynamic protein turnover,” Analytical Chemistry, Vol. 76, pp. 86-97 (2004)) used pulse labeling with ¹³C to measure protein turnover kinetics although the use of high atom percentages of ¹³C lead to dramatically extended isotope distributions that in the proteomics context would result in dramatic loss of separation space and, as is apparent in the Figures presented, the appearance of peaks at every unit across the mass spectrum. The data presented in FIG. 1 and FIG. 2 show similar features when the isotope ratio is altered too dramatically and emphasize the benefits of subtle modification of the isotope ratio. Based upon the data presented, it is suggested that doubling the ¹³C/¹²C ratio provides adequate isotope coding with minimal extension of the isotopomer distribution. Others have pointed out the benefits of ¹³C depletion (Marshall et al., “Protein molecular weight to 1 Da by ¹³C, ¹⁵N double-depletion and FT-ICR mass spectrometry,” Journal of the American Chemical Society, Vol. 119, pp. 433-434 (1997); Pasa-Tolic et al., 1999) and this strategy may be beneficial in the context of SMIRP.

For SMIRP to be useful in the context of expression proteomics it is necessary to control a number of variables such that significant changes in relative expression can be measured with quantifiable error. As the results show, there is significant variability associated with using relative isotopomer abundance for calculation of isotope ratio. While not wishing to be bound by any particular theory, it is believed that the origin of this variability is due to variability of relative isotopomer measurement by the mass spectrometer. Variability among different peptides as a result of their specific elemental compositions combined with metabolic bias, and natural variability of specific peptide isotope ratio based upon changes in flux that alter metabolic bias might also be significant in the context of proteomics. Measurement variability can be addressed by increased sampling and the use of mass spectrometers with improved sensitivity and resolution while the later natural variability must be explored in detail in future research. Thus, natural variability may provide additional information with respect to cellular flux in the future. SMIRP technology can be applied to any conceivable proteomics experiment including 2D gels, MuDPIT (Washburn et al., “Large-scale analysis of the yeast proteome by multidimensional protein identification technology,” Nature Biotechnology, Vol. 19, pp. 242-247 (2001)), accurate mass and time tags (Strittmatter et al, “Proteome analyses using accurate mass and elution time peptide tags with capillary LC time-of-flight mass spectrometry,” Journal of the American Society for Mass Spectrometry, Vol. 14, pp. 980-991 (2003)) and SILAC (Ong et al., “Stable isotope labeling by amino acids in cell culture, SILAC, as a simple and accurate approach to expression proteomics,” Molecular and Cellular Proteomics, Vol. 1, pp. 376-386 (2002)).

### EXAMPLES

The following examples illustrate a method of performing subtle isotope modification and monitoring peptide turnover. Modifications of these examples will be readily apparent to those skilled in the art seeking to perform isotope modification for expression proteomics or other applications differing from those described herein. These examples are included merely for purposes of illustration.

### Example 1

### Culture of Cells

Synechocystis sp. PCC 6803 cells were grown autotrophically in liquid BG-11 medium (available from Sigma; St. Louis, Mo.) (Rippka et al., “Generic assignments, strain histories and properties of pure cultures of cyanobacteria,” Journal of General Microbiology, Vol. 111, pp. 1-61 (1979)) buffered with 5 mM N-tris-hydroxymethyl-2-aminoethanesulfonic acid (TES)-NaOH (pH 8.0) (available from Sigma) and supplemented with filter-sterilized mixture of NaH¹³CO₂ (obtained from Cambridge Isotope Laboratories; 99% ¹³C; Andover, Mass.) and NaH¹²CO₂ (obtained from Sigma, 1.1% of ¹³C). The bicarbonate mixture was added to the freshly autoclaved BG-11 medium, which contained essentially no dissolved CO₂, and the bottles with the medium were sealed until further usage. The added NaH¹³CO₃ was calculated to be 0%, 1.5%, 3.0%, or 6.0% of the total NaHCO₃ taking into account natural ¹³C abundance (˜1 ¹³C/100 ¹²C) of the standard BG-11 medium (20 mg/l). Cultures were started by inoculating 50 ml of the medium (in a 150 ml flask) with a small amount of cells followed by incubation at 28° C. with light intensity of 50 mol photons/m² s while shaking at 100 rpm on a rotary shaker. Every 2-3 days the cultures were transferred to larger flasks and diluted with fresh BG-11 medium containing an appropriate amount of NaH¹³CO₃. The cultivation continued until the cell cultures reached OD₇₃₀=0.5 in a total volume of 500 ml. After that, cells were harvested by centrifugation, washed with thylakoid buffer (50 mM MES-NaOH at pH 7.0, 5 mM CaCl₂, 5 mM MgCl₂, 10 mM NaCl, 15% v/v glycerol, and 0.5% v/v DMSO), and then frozen in liquid nitrogen.

### Example 2

### Protein Preparation

Cells were thawed rapidly and placed on ice. Protease inhibitors (obtained from Sigma; P8465; 50 1/1 ml aliquot) were added prior to transfer of the cell suspension to tubes containing glass beads (0.1 mm; 1.0-1.2 g) pre-cooled on ice. Cells were broken using a micro-beadbeater (obtained from Biospec Products; Bartlesville, Okla.) on its maximum setting (4-5×30 s). Cells were cooled on ice between each treatment. Cell breakage efficiency was assessed by extraction of cells in acetone (10 μl cells plus 1 ml 80% acetone), agitation and centrifugation; chlorophyll was only extracted after cell breakage yielding a blue pellet. The broken cell suspension was diluted 10-fold with ice cold thylakoid buffer containing protease inhibitors and decanted to pre-cooled centrifuge tubes. Unbroken cells were removed (500 rpm SS34; 1 min) prior to transfer to clean tubes and sedimentation of the membranes (20,000 rpm SS34; 30 min). The supernatant was retained for soluble proteins and the pellet was resuspended in thylakoid buffer, homogenized (Teflon/glass) and stored at −80° C.

### Example 3

### Liquid Chromatography Electrospray-Ionization Mass Spectrometry with Fraction Collection

Samples of Synechocystis membranes were analyzed by LCMS+ (Whitelegge et al., 2002). Membrane fraction proteins (300-600 g) were precipitated at the interface of an aqueous chloroform/methanol phase separation (Wessel and Flugge, “A method for the quantitative recovery of protein in dilute solution in the presence of detergents and lipids.,” Analytical Biochemistry, Vol. 138, pp. 141-43 (1984)) as described (Whitelegge et al., “Toward the bilayer proteome, electrospray ionization-mass spectrometry of large, intact transmembrane proteins,” Proceedings of the National Academy of Sciences of the United States of America, Session 96, pp. 10695-0698 (1999)). Precipitated proteins were recovered after removal of the aqueous phase and addition of methanol. Precipitated samples were dried at atmospheric pressure for 2 min (25° C.) and dissolved in 90% formic acid (available from Sigma; 100 μl) immediately prior to HPLC. Reverse phase chromatography (RPC) of intact proteins was performed as described previously (Whitelegge et al., 2002; Whitelegge, “Thylakoid membrane proteomics,” Photosynthesis Research, Vol. 78, pp. 265-277 (2003); and Whitelegge, “HPLC and mass spectrometry of intrinsic membrane proteins,” Methods in Molecular Biology, Vol. 251, pp. 323-340 (2004)) using a macroporous polymeric support (obtained Polymer Labs; Amherst, Mass.; PLRP/S, 300 A, 5 m, 2×150 mm) at 100 μl/min (40° C.). The column was previously equilibrated in 95% A, 5% B (A, 0.1% TFA in water; B, 0.05% TFA in acetonitrile/isopropanol, 1:1, v/v) and eluted with a compound linear gradient from 5% B at 5 min after injection, through 40% B at 30 minutes and to 100% B at 150 min. The eluent was passed through a UV detector (280 nm) prior to a liquid-flow splitter (inserted between HPLC detector and mass spectrometer) that made it possible to collect fractions concomitant with electrospray-ionization mass spectrometry (ESI-MS). Fused silica capillary was used to transfer liquid to the ESI source (˜50 cm) or fraction collector (˜25 cm). The split fractions were collected into micro-centrifuge tubes at 1 min intervals. ESI-MS was performed as described (Whitelegge et al., “Electrosprayionization mass spectrometry of intact intrinsic membrane proteins,” Protein Science, Vol. 7, pp. 1423-430 (1998)) using a triple quadruple instrument (obtained from Applied Biosystems; Foster City, Calif.; API III). Orifice voltage was ramped from 60 to 120 over the mass range acquired (600-2300) and the instrument scanned with a step size of 0.3 amu and 1 ms dwell. Data were processed using MacSpec 3.3, Hypermass or BioMultiview 1.3.1 software (obtained from Applied Biosystems).

### Example 4

### Trypsin Digestion

Selected fractions collected during LCMS+ were reduced, alkylated and treated with trypsin (obtained from Promega; Madison, Wis.; sequencing grade modified by reductive methylation). DTT (15 μl, 10 mM in 50 mM ammonium bicarbonate; 30 min, 24° C.) then iodoacetamide (15 μl, 55 mM in 50 mM ammonium bicarbonate; 20 min, 24° C.) and finally trypsin (12.5 μl, 6 ng/l in 50 mM ammonium bicarbonate; 3 h, 37° C.) was added to aliquots of fractions (10 μl). After incubation, samples were dried by centrifugal evaporation and stored at −20° C. prior to analysis by LC-MSMS.

### Example 5

### Mass Spectrometry by MALDI-TOF (Matrix-Assisted Laser Desorption Ionization Time of-Flight) Mass Spectrometry

Dried reaction mixtures were re-dissolved in 5 μl of 70% acetic acid and analyzed (0.2 μl plus 0.5 μl matrix) by matrix-assisted laser desorption ionization (MALDI) coupled to delayed extraction time-of-flight MS in the reflector mode (obtained from Applied Biosystems; Voyager DE STR) using -cyano-4-hydroxycinnamic acid as matrix (10 mg/ml solution in water/acetonitrile/TFA 30/70/0.1) and internal/external calibration with bovine insulin. Manufacturer supplied default settings optimized for peptides less than 6000 Da were used for all samples.

### Example 6

### LC-MSMA (Micro-Liquid Chromatography with Tandem Mass Spectrometry)

Samples were analyzed by LC-MSMS with data-dependent acquisition (obtained from ThermoFinnigan; San Jose, Calif.; LCQ-DECA) after dissolution in 5 μl of 70% acetic acid (v/v). A reverse phase column (obtained from Michrom Biosciences, San Jose, CA; 200 m×10 cm; PLRP/S 5 m, 300 Å) was equilibrated for 10 min at 1.5 μl/min with 95% A, 5% B (A, 0.1% formic acid in water; B, 0.1% formic acid in acetonitrile) prior to sample injection. A linear gradient was initiated 10 min after sample injection ramping to 60% A, 40% B after 50 min and 20% A, 80% B after 65 min. Column eluent was directed to a coated glass electrospray emitter (TaperTip, TT150-50-50-CE-5, New Objective) at 3.3 kV for ionization without nebulizer gas. The mass spectrometer was operated in ‘triple-play’ mode with a survey scan (400-500 m/z), data-dependent zoom scan and MSMS. Individual sequencing experiments were matched to a custom Synechocystis sequence database using Sequest software (obtained from ThermoFinnigan).

### Example 7

### Calculation of Isotope Ratio from Peptide Distribution (ISOSOLV)

For a carbon isotope distribution the probability of having ‘n’ ¹³C's given ‘X’ total carbons and a ¹³C probability of ‘P’ is described as follows:

prob(n)=combin(X,n)*Pn*(1−P)(X−n)

When given an elemental composition Isosolv uses this for estimation of ¹³C/¹²C ratio. When given a molecular weight, the number of carbons is estimated by dividing the molecular weight by 110 (the average mass of an amino acid) and multiplying by 4.94 (the average number of carbons per amino acid). Then, given a measured isotopic distribution, the ¹³C probability can be determined by calculating the difference between the measured distribution and the theoretical distribution for an arbitrary ¹³C abundance. The estimated ¹³C abundance parameters are then incrementally altered until the error between the theoretical distribution and the calculated distribution has been minimized thus yielding the ¹³C probability in the measured spectrum. The version of Isosolv used in these examples includes natural minor contributions of D, ¹⁵N, ¹⁷/¹⁸O only.

While the description above refers to particular embodiments of the present invention, it will be understood that many modifications may be made without departing from the spirit thereof. The accompanying claims are intended to cover such modifications as would fall within the true scope and spirit of the present invention. The presently disclosed embodiments are therefore to be considered in all respects as illustrative and not restrictive, the scope of the invention being indicated by the appended claims, rather than the foregoing description, and all changes that come within the meaning and range of equivalency of the claims are therefore intended to be embraced therein.

What is claimed is:

1. A method for performing expression proteomic analysis, comprising:
inducing a subtle isotope modification in an organism or sample;
analyzing said subtle isotope modification with an analytic tool.

2. The method of claim 1 wherein said analytic tool is used to monitor turnover of peptides, polypeptides or both.

3. The method of claim 1 wherein said analytic tool is configured to calculate an isotope ratio for specific peptides or polypeptides by measuring isotope distributions.

4. The method of claim 1 wherein said organism is a mammal.

5. The method of claim 1 wherein said organism is a human.

6. The method of claim 1 wherein said subtle isotope modification includes swapping of ¹³C for ¹²C, ¹⁸O for ¹⁶O, ¹⁵N for ¹⁴N, deuterium for hydrogen, or combinations thereof.

7. The method of claim 1 wherein said subtle isotope ratio modification results in a modification of a ¹³C:¹²C isotope ratio of from about 100:1 to about 200:1.

8. The method of claim 1 wherein said subtle isotope modification is induced by a method selected from the group consisting of isotope-coded affinity tag (ICAT), stable isotope labeling with amino acids in cell culture (SILAC), enzymatic exchange, growth in stable isotopes, diet, injection and combinations thereof.

9. The method of claim 1 wherein said subtle isotope modification is analyzed by a method selected from the group consisting of matrix-assisted laser desorption ionization time-of-flight (MALDI-TOF) mass spectrometry, micro-liquid chromatography with tandem mass spectrometry (LC-MSMS), high resolution mass spectrometry, tandem mass spectrometry (MSMS), Fournier transform mass spectrometry (FTMS), averagine, isotope ratio mass spectrometry (IRMS) and combinations thereof.

10. A system for analyzing isotope distribution, comprising:
an organism in which a subtle isotope modification has been induced; and
an analytic tool to analyze said subtle isotope modification.

11. The system of claim 10 wherein said analytic tool is configured to analyze turnover of peptides, polypeptides or both.

12. The system of claim 10 wherein said analytic tool is configured to calculate an isotope ratio for specific peptides or polypeptides by measuring isotope distributions.

13. The system of claim 10 wherein said organism is a mammal.

14. The system of claim 10 wherein said organism is a human.

15. The system of claim 10 wherein said subtle isotope modification includes swapping of ¹³C for ¹²C, ¹⁸O for ¹⁶O, ¹⁵N for ¹⁴N, deuterium for hydrogen, or combinations thereof.

16. The system of claim 10 wherein said subtle isotope ratio modification results in a modification of a ¹³C:¹²C isotope ratio of from about 100:1 to about 200:1.

17. The system of claim 10 wherein said subtle isotope modification is induced by a method selected from the group consisting of isotope-coded affinity tag (ICAT), stable isotope labeling with amino acids in cell culture (SILAC), enzymatic exchange, growth in stable isotopes, diet, injection and combinations thereof.

18. The system of claim 10 wherein said analytic tool is configured to analyze said subtle isotope modification by a method selected from the group consisting of matrix-assisted laser desorption ionization time-of-flight (MALDI-TOF) mass spectrometry, micro-liquid chromatography with tandem mass spectrometry (LC-MSMS), high resolution mass spectrometry, tandem mass spectrometry (MSMS), Fournier transform mass spectrometry (FTMS), averagine, isotope ratio mass spectrometry (IRMS) and combinations thereof.

19. A method for performing expression proteomic analysis, comprising:
providing an elemental composition in which a subtle isotope modification has been induced;
analyzing said subtle isotope modification with the Isosolv algorithm.

20. The method of claim 19 wherein said subtle isotope modification includes swapping of ¹³C for ¹²C, ¹⁸O for ¹⁶O, ¹⁵N for ¹⁴N, deuterium for hydrogen, or combinations thereof.

21. The method of claim 19 wherein said subtle isotope ratio modification results in a modification of a ¹³C:¹²C isotope ratio of from about 100:1 to about 200:1.

22. The method of claim 19 wherein said subtle isotope modification is induced by a method selected from the group consisting of isotope-coded affinity tag (ICAT), stable isotope labeling with amino acids in cell culture (SILAC), enzymatic exchange, growth in stable isotopes, diet, injection and combinations thereof.

23. The method of claim 19 wherein said subtle isotope modification is analyzed by a method selected from the group consisting of matrix-assisted laser desorption ionization time-of-flight (MALDI-TOF) mass spectrometry, micro-liquid chromatography with tandem mass spectrometry (LC-MSMS), high resolution mass spectrometry, tandem mass spectrometry (MSMS), Fournier transform mass spectrometry (FTMS), averagine, isotope ratio mass spectrometry (IRMS) and combinations thereof.
